(19) United States
(12) Patent Application Publication
(10) Pub. No.: US 2025/0363767 A1
(43) Pub. Date: Nov. 27, 2025

(54) INFORMATION ASSISTANCE DEVICE, ENDOSCOPE SYSTEM, INFORMATION ASSISTANCE METHOD, AND COMPUTER-READABLE RECORDING MEDIUM

(71) Applicant: OLYMPUS CORPORATION, Tokyo (JP)

(72) Inventors: Kazumasa KUNUGI, Tokyo (JP); Ryuichi YORIMOTO, Tokyo (JP)

(73) Assignee: OLYMPUS CORPORATION, Tokyo (JP)

(21) Appl. No.: 18/969,586

(22) Filed: Dec. 5, 2024

(30) Foreign Application Priority Data
May 27, 2024 (JP) 2024-085747

(51) Int. Cl. G06V 10/25; A61B 1/04

(52) U.S. Cl. CPC G06V 10/25; A61B 1/04; G06V 2201/07

(57) ABSTRACT

An information assistance device includes: a processor including hardware, the processor being configured to set a specific region in an endoscopic image captured by an endoscope device as a region of interest, recognize an attention site captured in the endoscopic image, recognize a treatment tool captured in the endoscopic image, calculate a distance between the attention site and the treatment tool, and transmit a signal to notify specific information when the distance is equal to or less than a specific threshold value and the attention site is located outside the region of interest.

### CROSS REFERENCES TO RELATED APPLICATIONS

This application is based upon and claims the benefit of priority from Japanese Patent Application No. 2024-085747, filed on May 27, 2024, the entire contents of which are incorporated herein by reference.

### BACKGROUND

### 1. Technical Field

The present disclosure relates to an information assistance device, an endoscope system, an information assistance method, and a computer-readable recording medium.

### 2. Related Art

In the related art, an information assistance system that supports an operator is known (see, for example, JP 2009-233240 A).

The information assistance system (surgery assistance system) described in JP 2009-233240 A supports an operator by issuing a warning notifying the operator that a treatment tool is approaching an attention site in a living tissue during surgery. The attention site corresponds to a dangerous site that the treatment tool should not contact.

### SUMMARY

In some embodiments, an information assistance device includes: a processor including hardware, the processor being configured to set a specific region in an endoscopic image captured by an endoscope device as a region of interest, recognize an attention site captured in the endoscopic image, recognize a treatment tool captured in the endoscopic image, calculate a distance between the attention site and the treatment tool, and transmit a signal to notify specific information when the distance is equal to or less than a specific threshold value and the attention site is located outside the region of interest.

In some embodiments, an endoscope system includes: the information assistance device; and an endoscope device configured to capture an image of an inside of a subject.

In some embodiments, provided is an information assistance method executed by an information assistance device. The information assistance method includes: setting a specific region in an endoscopic image captured by an endoscope device as a region of interest; recognizing an attention site captured in the endoscopic image; recognizing a treatment tool captured in the endoscopic image; calculating a distance between the attention site and the treatment tool; and transmitting a signal to notify specific information when the distance is equal to or less than a specific threshold value and the attention site is located outside the region of interest.

In some embodiments, provided is a non-transitory computer-readable recording medium with an executable program stored thereon. The program causes an information assistance device to execute: setting a specific region in an endoscopic image captured by an endoscope device as a region of interest; recognizing an attention site captured in the endoscopic image; recognizing a treatment tool captured in the endoscopic image; calculating a distance between the attention site and the treatment tool; and transmitting a signal to notify specific information when the distance is equal to or less than a specific threshold value and the attention site is located outside the region of interest.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a block diagram illustrating a configuration of an endoscope system according to an embodiment;

FIG. 2 is a block diagram illustrating functions of a second processor;

FIG. 3 is a flowchart illustrating an information assistance method according to the embodiment;

FIG. 4 is a diagram illustrating an information assistance method;

FIG. 5 is a diagram illustrating an information assistance method;

FIG. 6 is a diagram illustrating an information assistance method;

FIG. 7 is a diagram illustrating a first modification of the embodiment;

FIG. 8 is a diagram illustrating a second modification of the embodiment;

FIG. 9 is a diagram illustrating the second modification of the embodiment;

FIG. 10 is a diagram illustrating the second modification of the embodiment;

FIG. 11 is a diagram illustrating a third modification of the embodiment; and

FIG. 12 is a diagram illustrating the third modification of the embodiment.

### DETAILED DESCRIPTION

Hereinafter, modes for carrying out the embodiments will be described with reference to the drawings. Note that the present invention is not limited by the embodiments described below. Furthermore, in the description of the drawings, the same portions are denoted by the same reference numerals.

### Configuration of Endoscope System

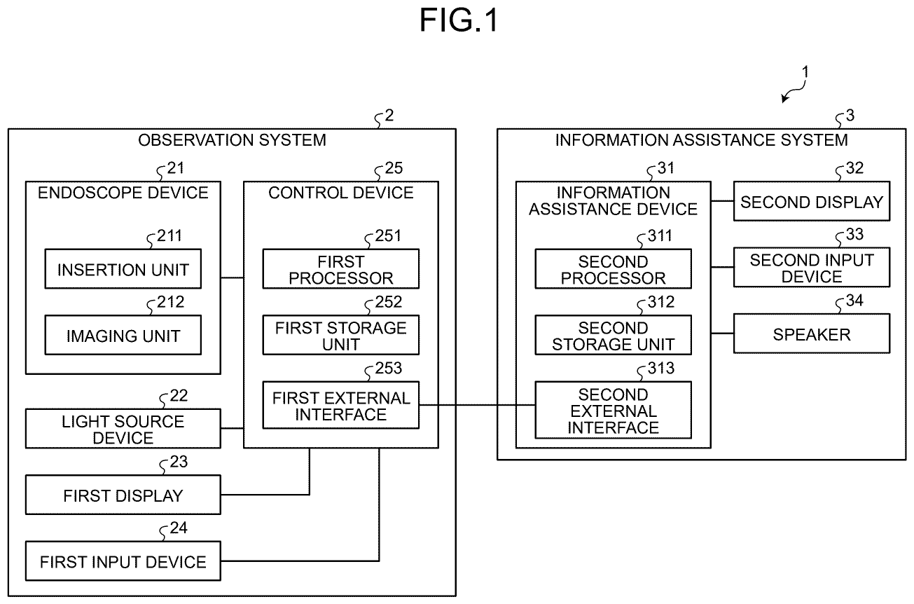

FIG. 1 is a block diagram illustrating a configuration of an endoscope system 1 according to an embodiment.

The endoscope system 1 is a system that is used in a medical field and provides information assistance to an operator while observing the inside of a subject. As illustrated in FIG. 1, the endoscope system 1 includes an observation system 2 and an information assistance system 3.

### Configuration of Observation System

The observation system 2 is a system that observes the inside of the subject. As illustrated in FIG. 1, the observation system 2 includes an endoscope device 21, a light source device 22, a first display 23, a first input device 24, and a control device 25.

As illustrated in FIG. 1, the endoscope device 21 includes an insertion unit 211 and an imaging unit 212.

The insertion unit 211 is configured by a so-called rigid endoscope. That is, the insertion unit 211 is rigid or at least partially flexible and has an elongated shape, and is inserted into the subject. Note that the endoscope device 21 according to the present embodiment includes a 3D scope. The insertion unit 211 includes a scope of a binocular relay system or a monocular pupil division system described below.

In the scope of the binocular relay system, two optical paths are arranged in parallel in the scope. In addition, an optical system is disposed in each of the two optical paths. Then, the scope of the binocular relay system takes in and emits right and left eye observation light having parallax with each other by the two optical systems (see, for example, JP 6-160731 A).

Furthermore, in the monocular pupil division type scope, one optical path is provided in the scope. In addition, an optical system is disposed in the one optical path.

Furthermore, a pupil division unit that divides a light flux in the pupil into two regions is provided at a pupil position of the optical system. Then, the scope of the monocular pupil division system takes in the observation light by the optical system, separates the observation light into right and left eye observation light having parallax with each other by the pupil division unit, and emits the observation light (see, for example, JP 6-59199 A).

Although not specifically illustrated, the imaging unit 212 includes a right-eye imaging unit that images the right-eye observation light (subject image) emitted from the insertion unit 211 and a left-eye imaging unit that images the left-eye observation light (subject image) emitted from the insertion unit 211. The right and left eye imaging units include a charge coupled device (CCD), a complementary metal oxide semiconductor (CMOS), or the like that receives the subject image and converts the subject image into an electrical signal, and generate an image signal by capturing the subject image. Note that, hereinafter, the image signal generated by the imaging unit 212 is referred to as a captured image.

The light source device 22 supplies observation light with which the subject is irradiated to a light guide (not illustrated) under the control of the control device 25. Examples of the observation light include white light including a visible wavelength band, excitation light for exciting a fluorescent agent such as indocyanine green, and narrow band light used in narrow band imaging (NBI). Then, the observation light supplied to the light guide is emitted to the subject from the distal end of the insertion unit 211 by passing through the insertion unit 211.

The first display 23 is a liquid crystal display (LCD), an electro luminescence (EL) display, or the like, and displays a display image generated by the control device 25 under the control of the control device 25.

The first input device 24 includes a keyboard, a mouse, a switch, a touch panel, and the like, and receives a user operation by a user such as an operator. Then, the first input device 24 outputs an operation signal corresponding to the user operation to the control device 25.

As illustrated in FIG. 1, the control device 25 includes a first processor 251, a first storage unit 252, and a first external interface 253.

The first processor 251 includes a controller such as a central processing unit (CPU) or a micro processing unit (MPU), or an integrated circuit such as an application specific integrated circuit (ASIC) or a field programmable gate array (FPGA), and controls the entire operation of the observation system 2. For example, the first processor 251 acquires a captured image generated by the imaging unit 212, and generates a display image (hereinafter, referred to as an endoscopic image) by applying predetermined image processing to the captured image. Then, the first processor 251 causes the first display 23 to display the endoscopic image. Furthermore, the endoscopic image is output to the outside via the first external interface 253.

The first storage unit 252 stores various programs executed by the first processor 251, information necessary for processing of the first processor 251, and the like.

The first external interface 253 is an interface that connects to an external device so as to be able to transmit and receive information.

Configuration of Information Assistance system

The information assistance system 3 is a system that supports a user such as an operator. As illustrated in FIG. 1, the information assistance system 3 includes an information assistance device 31, a second display 32, a second input device 33, and a speaker 34.

As illustrated in FIG. 1, the information assistance device 31 includes a second processor 311, a second storage unit 312, and a second external interface 313.

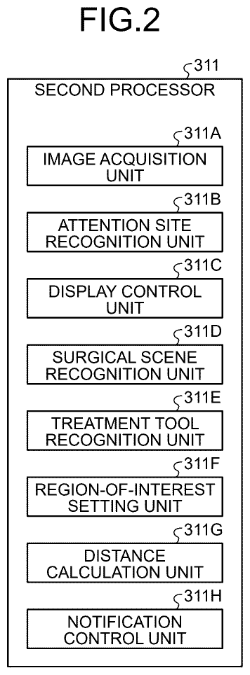

FIG. 2 is a block diagram illustrating functions of the second processor 311.

The second processor 311 includes a controller such as a CPU or an MPU, or an integrated circuit such as an ASIC or an FPGA, and controls the entire operation of the information assistance system 3. As illustrated in FIG. 2, the second processor 311 has functions of an image acquisition unit 311A, an attention site recognition unit 311B, a display control unit 311C, a surgical scene recognition unit 311D, a treatment tool recognition unit 311E, a region-of-interest setting unit 311F, a distance calculation unit 311G, and a notification control unit 311H. Details of each of the functions 311A to 311H in the second processor 311 will be described in the “information assistance method” described later.

The second storage unit 312 stores various programs (including the information assistance program) executed by the second processor 311, information necessary for processing of the second processor 311, and the like. Note that, as the information necessary for the processing of the second processor 311, the first to third learned models, the first and second relationship information, and the like can be exemplified. Details of the first to third learned models and the first and second relationship information will be described in the “information assistance method” described later.

The second external interface 313 is an interface that connects to an external device so as to be able to transmit and receive information. For example, the second external interface 313 receives the endoscopic image from the observation system 2 by way of the first external interface 253.

The second display 32 is an LCD, an EL display, or the like, and displays a display image (hereinafter, referred to as an information assistance image) generated by the display control unit 311C under the control of the display control unit 311C.

The second input device 33 includes a keyboard, a mouse, a switch, a touch panel, and the like, and receives a user operation by a user such as an operator. Then, the second input device 33 outputs an operation signal corresponding to the user operation to the information assistance device 31.

The speaker 34 corresponds to a notification portion. Then, the speaker 34 notifies specific information by voice under the control of the notification control unit 311H.

### Information Assistance Method

Next, an information assistance method executed by the information assistance device 31 will be described.

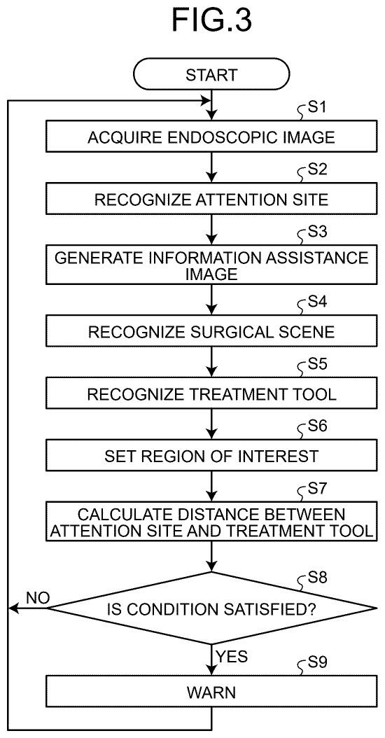

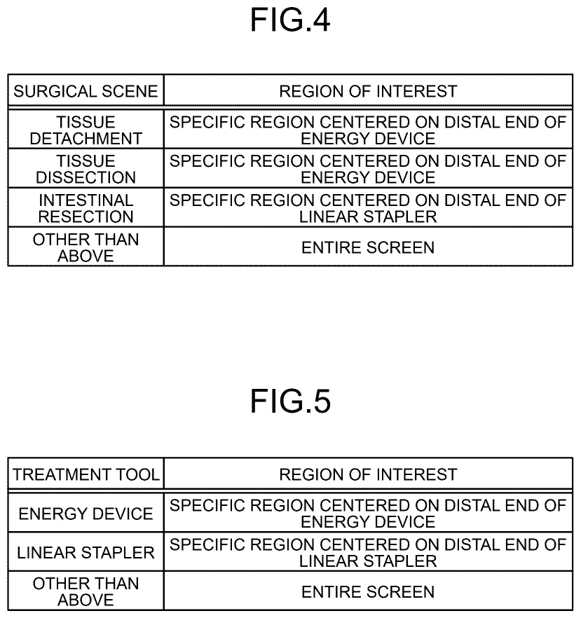

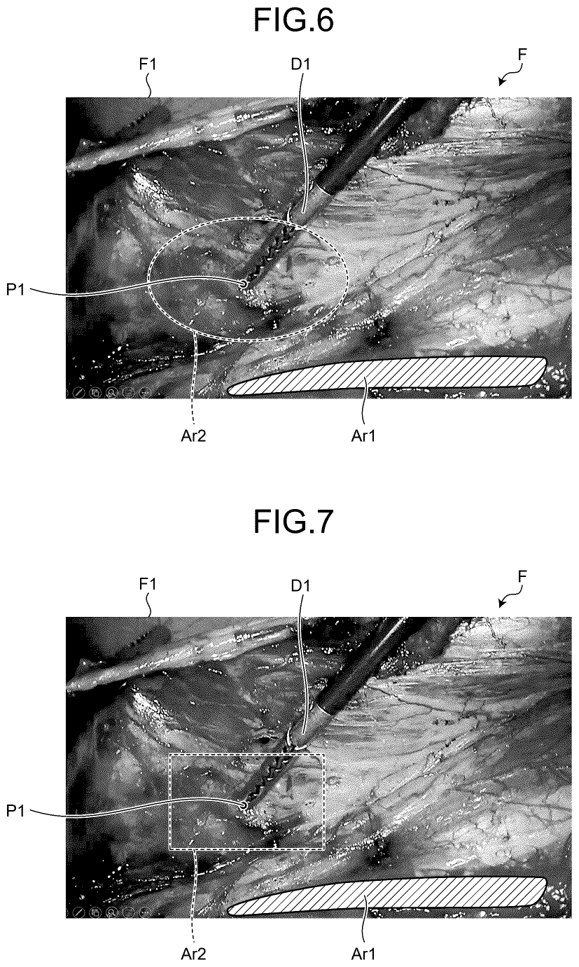

FIG. 3 is a flowchart illustrating an information assistance method according to the embodiment. FIGS. 4 to 6 are diagrams illustrating the information assistance method. FIG. 4 is a diagram illustrating first relationship information. FIG. 5 is a diagram illustrating second relationship information. FIG. 6 is a diagram illustrating an example of information assistance image F displayed on the second display 32.

Note that the endoscope system 1 has already been set to the following states.

That is, the insertion unit 211 is inserted into the subject, and an observation region of the observation system 2 is a region in the subject. That is, the first display 23 displays an endoscopic image generated by the imaging unit 212 and subjected to predetermined image processing by the first processor 251. Then, the operator performs treatment on the living tissue in the subject while confirming the endoscopic image displayed on the first display 23.

First, the image acquisition unit 311A acquires an endoscopic image from the observation system 2 via the first and second external interfaces 253 and 313 (Step S1).

After Step S1, the attention site recognition unit 311B recognizes the attention site captured in the endoscopic image by executing the estimation processing on the endoscopic image acquired in Step S1 using the first learned model stored in the second storage unit 312 (Step S2). In the case of large intestine surgery, examples of the attention site include ureter, hypogastric nerve, artery, and the like.

Here, the first learned model is a learning model after learning in which learning processing for the learning model is repeatedly executed using a plurality of sets of training images and teacher data each including a set of training images and teacher data. The training image is a captured image obtained by imaging the inside of the subject. The teacher data is data obtained by annotating the type, correct position, and size of the attention site in the training image. The learning model used for the learning processing is, for example, a convolutional neural network (CNN). Then, the first learned model includes a weight file (learning parameter) having a weight value and a bias value of the CNN.

After Step S2, the display control unit 311C generates the information assistance image F in which the region of the attention site recognized in Step S2 in the endoscopic image is given a specific color with respect to the endoscopic image acquired in Step S1 (Step S3). In the information assistance image F illustrated in FIG. 6, the region Ar1 of the attention site in the endoscopic image F1 is hatched for convenience of description. Then, the information assistance image F is displayed on the second display 32.

After Step S3, the surgical scene recognition unit 311D recognizes the current surgical scene performed by the operator by executing estimation processing on the endoscopic image acquired in Step S1 using the second learned model stored in the second storage unit 312 (Step S4).

Here, the second learned model is a learning model after learning in which learning processing for the learning model is repeatedly executed using a plurality of sets of training images and teacher data each including a set of training images and teacher data. The training image is a captured image obtained by imaging the inside of the subject. The teacher data is data that is performed when the training image is captured and annotates the type of surgical scene in the training image. The learning model used for the learning processing is, for example, a CNN. Then, the second learned model includes a weight file (learning parameter) having a weight value and a bias value of the CNN.

After Step S4, the treatment tool recognition unit 311E recognizes the treatment tool captured in the endoscopic image by executing the estimation processing on the endoscopic image acquired in Step S1 using the third learned model stored in the second storage unit 312 (Step S5). Note that examples of the treatment tool include an energy device that detaches or dissects a living tissue by applying ultrasonic vibration, high frequency current, heat, and the like to the living tissue, a linear stapler that sutures the living tissue, a gripping forceps that grips the living tissue, and the like.

Here, the third learned model is a learning model after learning in which learning processing for the learning model is repeatedly executed using a plurality of sets of training images and teacher data each including a set of training images and teacher data. The training image is a captured image obtained by imaging the inside of the subject. The teacher data is data obtained by annotating the type, correct position, and size of the treatment tool in the training image. The learning model used for the learning processing is, for example, a CNN. Then, the third learned model includes a weight file (learning parameter) having a weight value and a bias value of the CNN.

Note that the neural network used in the learning processing when generating the first to third learned models described above is not limited to the CNN, and other neural networks may be adopted. For example, a neural network such as a deep neural network (DNN), a transformer, or a generic adversarial network (GAN) may be appropriately adopted. In addition, as an algorithm of machine learning in the neural network, various known learning algorithms can be adopted. For example, a supervised learning algorithm using an error back propagation method can be adopted.

After Step S5, the region-of-interest setting unit 311F sets a specific region in the endoscopic image acquired in Step S1 as the region of interest (Step S6).

Specifically, in Step S6, the region-of-interest setting unit 311F uses the first relationship information or the second relationship information stored in the second storage unit 312.

In a case where the setting mode of the region of interest is set to the first setting mode by an input operation to the second input device 33 by a user such as an operator, the region-of-interest setting unit 311F uses the first relationship information.

Here, as illustrated in FIG. 4, the first relationship information is information in which the surgical scene and the region of interest are associated with each other. Specifically, the surgical scenes of “tissue detachment” and “tissue dissection” are respectively associated with regions of interest in “specific regions centered on the distal end of the energy device”. In addition, the surgical scene of “intestinal resection” is associated with a region of interest in a “specific region centered on the distal end of the linear stapler”. Moreover, surgical scenes other than “tissue detachment”, “tissue dissection”, and “intestinal resection” are associated with regions of interest in the “entire screen”. In the present embodiment, the distal end of the treatment tool (for example, an energy device, a linear stapler, or the like) is, for example, the earliest end point on the treatment performing side in the treatment tool. Note that the distal end of the treatment tool is not limited thereto, and a predetermined point at the end portion of the treatment tool on the treatment side can be set.

For example, it is assumed that “tissue detachment” or “tissue dissection” is recognized as the surgical scene in Step S4.

In this case, in Step S6, the region-of-interest setting unit 311F refers to the first relationship information and specifies the region of interest of the “specific region centered on the distal end of the energy device” corresponding to the surgical scene of the “tissue detachment” or the “tissue dissection”. Then, in the endoscopic image acquired in Step S1, the region-of-interest setting unit 311F sets a specific region centered on the distal end of the energy device that is the treatment tool recognized in Step S5 as the region of interest. In the information assistance image F illustrated in FIG. 6, for convenience of description, an elliptical region Ar2 centered on the distal end P1 of the energy device D1 captured in the endoscopic image F1 is illustrated as a region of interest. In the information assistance image F, the elliptical region Ar2 is not actually displayed.

Furthermore, for example, a case where “intestinal resection” is recognized as the surgical scene in Step S4 is assumed.

In this case, in Step S6, the region-of-interest setting unit 311F refers to the first relationship information and specifies the region of interest of the “specific region centered on the distal end of the linear stapler” corresponding to the surgical scene of the “intestinal resection”. Then, in the endoscopic image acquired in Step S1, the region-of-interest setting unit 311F sets a specific region centered on the distal end of the linear stapler, which is the treatment tool recognized in Step S5, as the region of interest.

Furthermore, for example, in Step S4, it is assumed that a scene other than “tissue detachment”, “tissue dissection”, and “intestinal resection” is recognized as the surgical scene.

In this case, in Step S6, the region-of-interest setting unit 311F refers to the first relationship information, and specifies the region of interest of the “entire screen” corresponding to the surgical scenes other than the “tissue detachment”, the “tissue dissection”, and the “intestinal resection”. Then, the region-of-interest setting unit 311F sets the entire endoscopic image as the region of interest in the endoscopic image acquired in Step S1.

Furthermore, in a case where the setting mode of the region of interest is set to the second setting mode by an input operation to the second input device 33 by a user such as an operator, the region-of-interest setting unit 311F uses the second relationship information.

Here, as illustrated in FIG. 5, the second relationship information is information in which the treatment tool and the region of interest are associated with each other. Specifically, the treatment tool of the “energy device” is associated with a region of interest in a “specific region centered on the distal end of the energy device”. In addition, the treatment tool of the “linear stapler” is associated with a region of interest of a “specific region centered on the distal end of the linear stapler”. Further, treatment tools other than the “energy device” and the “linear stapler” are associated with a region of interest in the “entire screen”.

For example, it is assumed that the “energy device” is recognized as the treatment tool in Step S5.

In this case, in Step S6, the region-of-interest setting unit 311F refers to the second relationship information and specifies the region of interest of the “specific region centered on the distal end of the energy device” corresponding to the treatment tool of the “energy device”. Then, in the endoscopic image acquired in Step S1, the region-of-interest setting unit 311F sets a specific region centered on the distal end of the energy device that is the treatment tool recognized in Step S5 as the region of interest. In the information assistance image F illustrated in FIG. 6, for convenience of description, an elliptical region Ar2 centered on the distal end P1 of the energy device D1 captured in the endoscopic image F1 is illustrated as a region of interest. In the information assistance image F, the elliptical region Ar2 is not actually displayed.

Furthermore, for example, a case where “linear stapler” is recognized as the treatment tool in Step S5 is assumed.

In this case, in Step S6, the region-of-interest setting unit 311F refers to the second relationship information and specifies the region of interest of the “specific region centered on the distal end of the linear stapler” corresponding to the treatment tool of the “linear stapler”. Then, in the endoscopic image acquired in Step S1, the region-of-interest setting unit 311F sets a specific region centered on the distal end of the linear stapler, which is the treatment tool recognized in Step S5, as the region of interest.

Furthermore, for example, it is assumed that a device other than the “energy device” and the “linear stapler” is recognized as the treatment tool in Step S5.

In this case, in Step S6, the region-of-interest setting unit 311F refers to the second relationship information and specifies the region of interest of the “entire screen” corresponding to the treatment tool other than the “energy device” and the “linear stapler”. Then, the region-of-interest setting unit 311F sets the entire endoscopic image as the region of interest in the endoscopic image acquired in Step S1.

Note that, in Step S5, a plurality of treatment tools may be recognized. In this case, in Step S6, the region-of-interest setting unit 311F refers to the second relationship information and specifies a region of interest corresponding to a treatment tool with high priority among the plurality of recognized treatment tools. The order of the higher priority is, for example, the order of “energy device”, “linear stapler”, and “other treatment tools”.

After Step S6, the distance calculation unit 311G calculates the distance between the actual position of the attention site on the observation target corresponding to the attention site recognized in Step S2 and the actual position of the treatment tool on the observation target corresponding to the treatment tool recognized in Step S5 (Step S7).

In the present embodiment, as described above, the endoscope device 21 includes a 3D scope. Therefore, in Step S7, the distance calculation unit 311G uses the relative shift amount in the image of the same subject between the respective captured images simultaneously captured from different viewpoints in the right and left eye imaging units of the imaging unit 212. Then, the distance calculation unit 311G calculates the three-dimensional coordinates (hereinafter, referred to as first three-dimensional coordinates) of the actual attention site on the observation target based on the principle of triangulation. Similarly, the distance calculation unit 311G calculates three-dimensional coordinates (hereinafter, referred to as second three-dimensional coordinates) of the actual treatment tool on the observation target based on the principle of triangulation. Then, the distance calculation unit 311G calculates, for example, the shortest distance between the actual position of the attention site on the observation target and the actual position of the treatment tool on the observation target based on the first and second three-dimensional coordinates. As the actual position of the treatment tool on the observation target, the position of the distal end of the treatment tool can be used.

After Step S7, the notification control unit 311H determines whether or not the condition is satisfied (Step S8). The condition is a condition that the distance calculated in Step S7 is equal to or less than a specific threshold value, and the attention site to be the target in calculating the distance is located outside the region of interest set in Step S6.

When it is determined that the condition is not satisfied (Step S8: No), the information assistance device 31 returns to Step S1.

Meanwhile, when it is determined that the condition is satisfied (Step S8: Yes), the notification control unit 311H causes the speaker 34 to notify specific information indicating a warning by voice (Step S9). Thereafter, the information assistance device 31 returns to Step S1.

In the example of FIG. 6, the region Ar1 of the attention site is located outside the region of interest (elliptical region Ar2). Then, in a case where the distance between the actual position of the attention site on the observation target corresponding to the region Ar1 of the attention site and the actual position of the distal end P1 on the observation target corresponding to the distal end P1 of the energy device D1 is equal to or less than a specific threshold value, specific information indicating a warning is notified by voice from the speaker 34.

According to the present embodiment described above, the following effects are obtained.

In the information assistance device 31 according to the present embodiment, the second processor 311 sets a specific region in the endoscopic image as the region of interest. In addition, the second processor 311 recognizes the attention site in the subject captured in the endoscopic image. Furthermore, the second processor 311 recognizes the treatment tool captured in the endoscopic image. In addition, the second processor 311 calculates the distance between the attention site and the treatment tool. Then, the second processor 311 causes the speaker 34 to report specific information in a case where the distance is equal to or less than a specific threshold value and the attention site is located outside the region of interest.

Therefore, a warning is not issued to the operator when the attention site exists in the region of interest, and the treatment by the operator is not hindered. In addition, since a warning is issued to the operator when the attention site exists outside the region of interest and the treatment tool approaches the attention site, it is possible to avoid bringing the treatment tool into contact with the attention site that the operator does not pay attention to.

Therefore, according to the information assistance device 31 according to the present embodiment, it is possible to implement information assistance that reduces the burden on the operator.

Furthermore, in the information assistance device 31 according to the present embodiment, the second processor 311 sets the region of interest based on the treatment tool captured in the endoscopic image. Specifically, a description will be given of a case where a treatment tool captured in an endoscopic image is an attention treatment tool (for example, an energy device or linear stapler) which has a problem when coming into contact with the attention site or in which a problem easily occurs. In this case, the second processor 311 sets a region centered on a specific position (the distal end in the present embodiment) of the attention treatment tool as the region of interest. Therefore, the region of interest can be set to an appropriate region. Furthermore, in a case where the treatment tool captured in the endoscopic image is other than the energy device and is other than the linear stapler, the second processor 311 sets the entire endoscopic image as the region of interest. That is, with respect to a treatment tool such as a gripping forceps that does not cause a problem even when the treatment tool comes into contact with the attention site or hardly causes a problem, since the entire endoscopic image is set as the region of interest, an unnecessary warning is not issued to the operator, and the treatment by the operator is not hindered.

Furthermore, in the information assistance device 31 according to the present embodiment, the second processor 311 recognizes a surgical scene based on an endoscopic image, and sets a region of interest based on the surgical scene. Specifically, when the surgical scene is a surgical scene of detaching tissue, a surgical scene of dissecting tissue, or a surgical scene of resecting an intestinal tract, the second processor 311 sets a region centered on a specific position (distal end in the present embodiment) of the energy device or the linear stapler as the region of interest. Therefore, the region of interest can be set to an appropriate region. In addition, the second processor 311 sets the entire endoscopic image as the region of interest in a case where the surgical scene is not a surgical scene in which the tissue is detached, a surgical scene in which the tissue is dissected, or a surgical scene in which the intestinal tract is resected. That is, for a surgical scene using the gripping forceps or the like in which there is no problem even when the treatment tool comes into contact with the attention site, the entire endoscopic image is set as the region of interest, and thus, an unnecessary warning is not issued to the operator, and a treatment by the operator is not hindered.

### Other Embodiments

Hereinbefore, the embodiment for carrying out the present invention is described. However, the present invention should not be limited only by the above-described embodiment.

In the above-described embodiment, the information assistance device is mounted in the endoscope system 1 in which the insertion unit 211 is configured by a rigid endoscope, but the embodiment is not limited thereto. For example, the information assistance device may be mounted on an endoscope system in which the insertion unit 211 is configured by a flexible endoscope.

In the above-described embodiment, since the endoscope device 21 is configured by the 3D scope, the distance calculation unit 311G calculates the distance between the actual position of the attention site on the observation target and the actual position of the treatment tool on the observation target based on the principle of triangulation, but the embodiment is not limited thereto.

For example, in a case where the endoscope device 21 is not the 3D scope, the distance calculation unit 311G may calculate the distance between the actual position of the attention site on the observation target and the actual position of the treatment tool on the observation target as described below.

The distance calculation unit 311G estimates the first and second three-dimensional coordinates by performing estimation processing using a specific learned model on the two-dimensional image generated by the imaging unit 212. Then, the distance calculation unit 311G calculates a distance between the actual position of the attention site on the observation target and the actual position of the treatment tool on the observation target.

Furthermore, in a case where the imaging element constituting the imaging unit 212 of the endoscope device 21 includes an imaging element including a phase difference pixel, the distance calculation unit 311G calculates the distance between the position of the actual attention site on the observation target and the position of the actual treatment tool on the observation target based on pixel information corresponding to the phase difference pixel.

Furthermore, the distance calculation unit 311G converts the number of pixels between the position of the attention site on the display image on the first display 23 or the second display 32 and the position of the treatment tool into a distance.

In the above-described embodiment, the display control unit 311C displays the information assistance image F on the second display 32, but the embodiment is not limited thereto. For example, the display control unit 311C may transmit the information assistance image F to the observation system 2 via the first and second external interfaces, and cause the first display 23 to display the information assistance image F.

By the way, when a specific time period elapses after the power supplied to the energy device is switched from on to off, the residual heat of the treatment unit of the energy device disappears, and there is no problem even when the treatment unit comes into contact with the attention site.

Therefore, in the above-described embodiment, the second processor 311 may switch the region of interest to the entire endoscopic image after a specific time period has elapsed since the power supplied to the energy device is switched from on to off.

By the way, in a case where the treatment tool captured in the endoscopic image is an energy device and the power supplied to the energy device is off, there is no problem even when the energy device comes into contact with the attention site.

Therefore, in the above-described embodiment, in a case where the treatment tool captured in the endoscopic image is an energy device and the power supplied to the energy device is off, the second processor 311 may set the entire endoscopic image to the region of interest.

In the above-described embodiment, the speaker 34 is employed as the notification portion, but the embodiment is not limited thereto. A notification portion other than the speaker 34 may be adopted as long as specific information can be notified by at least one of an image, light, sound, and vibration. For example, the first display 23 or second display 32 can be exemplified as the notification portion that notifies specific information by an image. Examples of the notification portion that notifies specific information by light include a light emitting diode (LED) provided adjacent to the second display 32 and the like. Furthermore, as a notification portion that notifies specific information by vibration, a vibration member provided in the insertion unit 211 or a portion to be gripped by the treatment tool can be exemplified.

Further, in the above-described embodiment, first to third modifications described below may be adopted.

### First Modification

FIG. 7 is a diagram illustrating a first modification of the embodiment. Specifically, FIG. 7 is a diagram corresponding to FIG. 6.

In the above-described embodiment, the elliptical region Ar2 is adopted as the region of interest, but the embodiment is not limited thereto. The shape of the region of interest is not limited to an ellipse, and other shapes may be used. At this time, the shape of the region of interest is preferably a shape that is rotationally symmetric at a specific angle with the center of the region of interest as the central axis. In the example of FIG. 7, a rectangular region Ar2 is adopted as the region of interest. Furthermore, the size of the region of interest in the endoscopic image F1 may be changeable by an input operation to the second input device 33 by a user such as an operator.

Even in a case where the configuration of the first modification described above is adopted, the same effects as those of the above-described embodiment are obtained.

### Second Modification

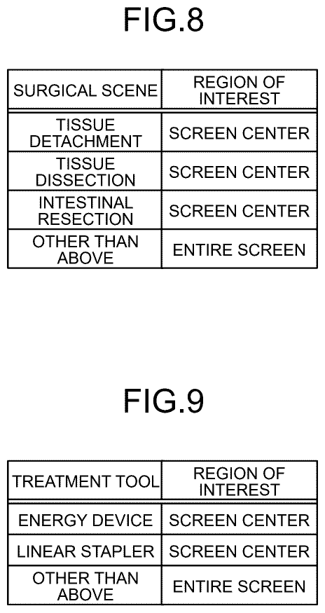

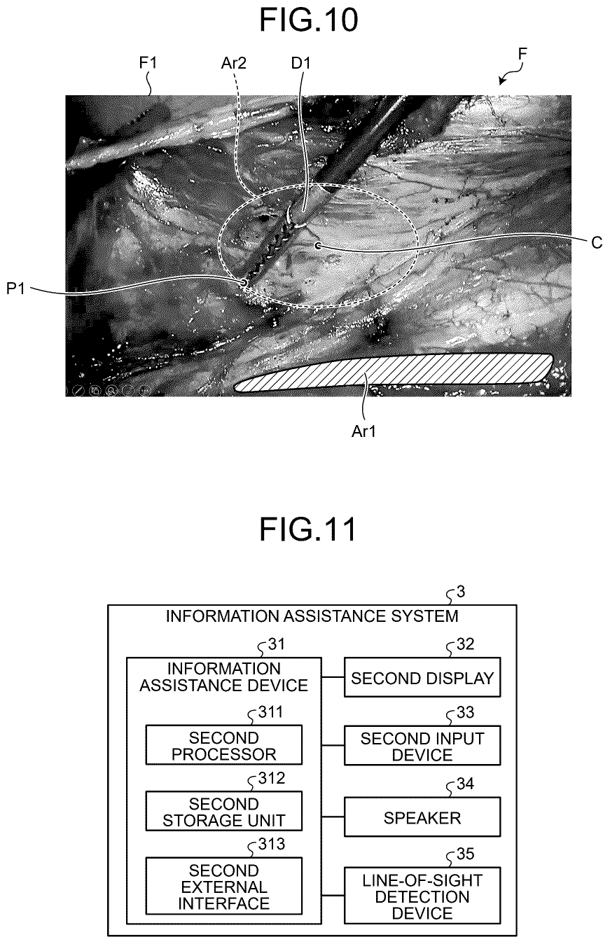

FIGS. 8 to 10 are diagrams illustrating a second modification of the embodiment. Specifically, FIG. 8 is a diagram corresponding to FIG. 4, and is a diagram illustrating first relationship information according to the second modification. FIG. 9 is a diagram corresponding to FIG. 5, and is a diagram illustrating second relationship information according to the second modification. FIG. 10 is a diagram corresponding to FIG. 6, and is a diagram illustrating an example of an information assistance image F according to the present second modification.

In the above-described embodiment, instead of the first and second relationship information illustrated in FIGS. 4 and 5, first and second relationship information according to the second modification illustrated in FIGS. 8 and 9 may be adopted.

The first relationship information according to the second modification is information in which a surgical scene and a region of interest are associated with each other, similarly to the above-described embodiment. Specifically, a surgical scene of attention (for example, surgical scenes of “tissue detachment”, “tissue dissection”, and “intestinal resection”) in which there is a problem or a problem is likely to occur when the treatment tool comes into contact with the attention site is associated with a region of interest in the “center of the screen” in which the operator is expected to pay attention. In addition, surgical scenes (a surgical scene in which there is no problem or a problem hardly occurs even when the treatment tool comes into contact with the attention site) other than “tissue detachment”, “tissue dissection”, and “intestinal resection” are associated with the region of interest of the “entire screen”.

For example, in Step S4, it is assumed that “tissue detachment”, “tissue dissection”, or “intestinal resection” is recognized as the surgical scene.

In this case, in Step S6, the region-of-interest setting unit 311F refers to the first relationship information, and specifies the region of interest in the “center of the screen” corresponding to the surgical scene of “tissue detachment”, “tissue dissection”, or “intestinal resection”. Then, the region-of-interest setting unit 311F sets a specific region centered on the screen center point of the endoscopic image acquired in Step S1 as the region of interest at the center of the screen. In the information assistance image F illustrated in FIG. 10, for convenience of description, the elliptical region Ar2 centered on a screen center point C of the endoscopic image F1 is illustrated as a region of interest. In the information assistance image F, the elliptical region Ar2 is not actually displayed.

Furthermore, for example, in Step S4, a case is assumed where a scene other than “tissue detachment”, “tissue dissection”, and “intestinal resection” is recognized as the surgical scene.

In this case, in Step S6, the region-of-interest setting unit 311F refers to the first relationship information, and specifies the region of interest of the “entire screen” corresponding to the surgical scenes other than the “tissue detachment”, the “tissue dissection”, and the “intestinal resection”. Then, the region-of-interest setting unit 311F sets the entire endoscopic image as the region of interest in the endoscopic image acquired in Step S1.

In addition, the second relationship information according to the second modification is information in which the treatment tool and the region of interest are associated with each other, similarly to the above-described embodiment. Specifically, the treatment tools of the “energy device” and the “linear stapler” are each associated with a region of interest in the “screen center” that the operator is expected to focus on. In addition, the treatment tool other than the “energy device” and the “linear stapler” is associated with the region of interest of the “entire screen”.

For example, it is assumed that an “energy device” or a “linear stapler” is recognized as the treatment tool in Step S5.

In this case, in Step S6, the region-of-interest setting unit 311F refers to the second relationship information and specifies the region of interest in the “center of the screen” corresponding to the treatment tool of the “energy device” or the “linear stapler”. Then, the region-of-interest setting unit 311F sets a specific region centered on the screen center of the endoscopic image acquired in Step S1 as the region of interest. In the information assistance image F illustrated in FIG. 10, for convenience of description, an elliptical region Ar2 centered on the screen center C of the endoscopic image F1 is illustrated as a region of interest. In the information assistance image F, the elliptical region Ar2 is not actually displayed.

In addition, for example, it is assumed that a device other than the “energy device” and the “linear stapler” is recognized as the treatment tool in Step S5. In this case, in Step S6, the region-of-interest setting unit 311F refers to the second relationship information and specifies the region of interest of the “entire screen” corresponding to the treatment tool other than the “energy device” and the “linear stapler”. Then, the region-of-interest setting unit 311F sets the entire endoscopic image as the region of interest in the endoscopic image acquired in Step S1.

Note that, in Step S5, a plurality of treatment tools may be recognized. In this case, in Step S6, the region-of-interest setting unit 311F refers to the second relationship information and specifies a region of interest corresponding to a treatment tool with high priority among the plurality of recognized treatment tools. The order of the higher priority is, for example, the order of “energy device”, “linear stapler”, and “other treatment tools”.

Even in a case where the configuration of the second modification described above is adopted, the same effects as those of the above-described embodiment are obtained.

In the second modification described above, in the first and second relationship information, the “center of the screen” is adopted as the region of interest corresponding to the surgical scene of the “tissue detachment”, the “tissue dissection”, or the “intestinal resection”, and the treatment tool of the “energy device” or the “linear stapler”, but the embodiment is not limited thereto. The surgical scene and the region of interest corresponding to the treatment tool are not limited to the screen center C, and may be a specific region centered on another position of the endoscopic image F1.

By the way, when a specific time period elapses after the power supplied to the energy device is switched from on to off, the residual heat of the treatment unit of the energy device disappears, and there is no problem even when the treatment unit comes into contact with the attention site.

Therefore, in the second modification described above, the second processor 311 may switch the region of interest to the entire endoscopic image after a specific time period has elapsed since the power supplied to the energy device is switched from on to off.

By the way, in a case where the treatment tool captured in the endoscopic image is an energy device and the power supplied to the energy device is off, there is no problem even when the energy device comes into contact with the attention site.

Therefore, in the second modification described above, in a case where the treatment tool captured in the endoscopic image is an energy device and the power supplied to the energy device is off, the second processor 311 may set the entire endoscopic image to the region of interest.

### Third Modification

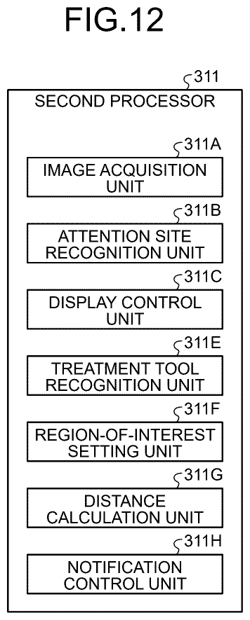

FIGS. 11 and 12 are diagrams illustrating a third modification of the embodiment. Specifically, FIG. 11 is a block diagram illustrating a configuration of an information assistance system 3 according to the third modification. FIG. 12 is a diagram corresponding to FIG. 2, and is a block diagram illustrating functions of a second processor 311 according to the third modification.

In the above-described embodiment, the method of setting the region of interest is not limited to the setting method described in the above-described embodiment, and as in the third modification, a specific region centered on the position (the position to which the operator pays attention) of the line of sight of the operator on the screen of the second display 32 may be set as the region of interest.

Specifically, in the information assistance system 3 according to the third modification, as illustrated in FIG. 11, a line-of-sight detection device 35 is added to the information assistance system 3 described in the above-described embodiment.

The line-of-sight detection device 35 is installed adjacent to the second display 32, such as below the second display 32. The line-of-sight detection device 35 includes a small camera or the like that photographs the eyeball from the front direction, and tracks the position of the pupil of the eyeball of the operator to detect, in real time, position coordinates (hereinafter, referred to as line-of-sight position coordinates) at which the line of sight of the operator is captured on the screen of the second display 32. Then, the line-of-sight detection device 35 outputs the line-of-sight position coordinates to the information assistance device 31.

In Step S6, the region-of-interest setting unit 311F according to the third modification sets a specific region centered on the line-of-sight position coordinates output from the line-of-sight detection device 35 in the endoscopic image as the region of interest. That is, in the third modification, the first and second relationship information are not stored in the second storage unit 312. Furthermore, as illustrated in FIG. 12, the second processor 311 according to the third modification does not have the function of the surgical scene recognition unit 311D with respect to the second processor 311 described in the above-described embodiment.

Even in a case where the configuration of the third modification described above is adopted, the same effects as those of the above-described embodiment are obtained.

According to an information assistance device, an endoscope system, an information assistance method, and a computer-readable recording medium according to the disclosure, it is possible to implement information assistance that reduces a burden on a doctor.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An information assistance device comprising:
a processor comprising hardware, the processor being configured to
set a specific region in an endoscopic image captured by an endoscope device as a region of interest,
recognize an attention site captured in the endoscopic image,
recognize a treatment tool captured in the endoscopic image,
calculate a distance between the attention site and the treatment tool, and
transmit a signal to notify specific information when the distance is equal to or less than a specific threshold value and the attention site is located outside the region of interest.

2. The information assistance device according to claim 1, wherein the processor is configured to set the region of interest based on the treatment tool captured in the endoscopic image.

3. The information assistance device according to claim 1, wherein the processor is configured to recognize a specific treatment tool captured in the endoscopic image among a plurality of treatment tools used during surgery.

4. The information assistance device according to claim 2, wherein the processor is configured to set a region centered on a specific position of an energy device as the region of interest when the treatment tool captured in the endoscopic image is the energy device.

5. The information assistance device according to claim 4, wherein the processor is configured to switch the region of interest to the entire endoscopic image after a specific time period has elapsed since power supplied to the energy device is switched from on to off.

6. The information assistance device according to claim 2, wherein the processor is configured to set the entire endoscopic image as the region of interest when the treatment tool captured in the endoscopic image is an energy device and power supplied to the energy device is off.

7. The information assistance device according to claim 2, wherein the processor is configured to set a region centered on a specific position of a linear stapler as the region of interest when the treatment tool captured in the endoscopic image is the linear stapler.

8. The information assistance device according to claim 2, wherein the processor is configured to set the entire endoscopic image as the region of interest when the treatment tool captured in the endoscopic image is other than an energy device and is other than a linear stapler.

9. The information assistance device according to claim 2, wherein the processor is configured to set a region centered on an image center of the endoscopic image as the region of interest when the treatment tool captured in the endoscopic image is either an energy device or a linear stapler.

10. The information assistance device according to claim 1, wherein the processor is configured to
recognize a surgical scene based on the endoscopic image, and
set the region of interest in the endoscopic image based on the surgical scene.

11. The information assistance device according to claim 10, wherein the processor is configured to set a region centered on a specific position of an energy device that is the treatment tool as the region of interest when the surgical scene is a surgical scene in which a tissue is detached or dissected.

12. The information assistance device according to claim 10, wherein the processor is configured to set a region centered on a specific position of a linear stapler that is the treatment tool as the region of interest when the surgical scene is a surgical scene in which an intestinal tract is resected.

13. The information assistance device according to claim 10, wherein the processor is configured to set the entire endoscopic image as the region of interest when the surgical scene is not a surgical scene in which a tissue is detached or dissected or a surgical scene in which an intestinal tract is resected.

14. The information assistance device according to claim 10, wherein the processor is configured to set a region centered on an image center of the endoscopic image as the region of interest when the surgical scene is a surgical scene in which a tissue is detached or dissected or a surgical scene in which an intestinal tract is resected.

15. The information assistance device according to claim 1, wherein the region of interest has a shape that is rotationally symmetric at a specific angle with a center of the region of interest as a central axis.

16. The information assistance device according to claim 1, wherein the processor is configured to transmit a signal to notify the specific information by at least one of an image, light, sound, and vibration.

17. An endoscope system comprising:
the information assistance device according to claim 1; and
an endoscope device configured to capture an image of an inside of a subject.

18. The endoscope system according to claim 17, further comprising:
a first display configured to display the endoscopic image; and
a second display configured to display the image processed by the information assistance device.

19. An information assistance method executed by an information assistance device, the information assistance method comprising:
setting a specific region in an endoscopic image captured by an endoscope device as a region of interest;
recognizing an attention site captured in the endoscopic image;
recognizing a treatment tool captured in the endoscopic image;
calculating a distance between the attention site and the treatment tool; and
transmitting a signal to notify specific information when the distance is equal to or less than a specific threshold value and the attention site is located outside the region of interest.

20. A non-transitory computer-readable recording medium with an executable program stored thereon, the program causing an information assistance device to execute:
setting a specific region in an endoscopic image captured by an endoscope device as a region of interest;
recognizing an attention site captured in the endoscopic image;
recognizing a treatment tool captured in the endoscopic image;
calculating a distance between the attention site and the treatment tool; and
transmitting a signal to notify specific information when the distance is equal to or less than a specific threshold value and the attention site is located outside the region of interest.
